(19) United States
(12) Patent Application Publication
(10) Pub. No.: US 2025/0152754 A1
(43) Pub. Date: May 15, 2025

(54) MICROBUBBLE LYOPHILIZED PREPARATION FOR ULTRASONIC RADIOGRAPHY, CONTRAST AGENT, AND PREPARATION METHOD

(71) Applicant: SHENZHEN XINQI PHARMACEUTICAL TECHNOLOGY CO., LTD., Shenzhen, Guangdong (CN)

(72) Inventor: Zhongwei ZHAO, Shenzhen, Guangdong (CN)

(21) Appl. No.: 18/695,231

(22) PCT Filed: Dec. 30, 2022
(86) PCT No.: PCT/CN2022/143941

§ 371 Date: Mar. 25, 2024

(30) Foreign Application Priority Data
Nov. 3, 2021 (CN) 202111296255.3

(51) Int. Cl. A61K 49/22; A61K 47/10

(52) U.S. Cl. CPC A61K 49/223; A61K 47/10

(57) ABSTRACT

A microbubble lyophilized preparation for ultrasonic radiography relates to the field of contrast agents. The lyophilized preparation has polyethylene glycol and a surfactant. The percentage of the folded polymer chain of the polyethylene glycol is less than 34%. When the percentage of the folded polymer chain of the polyethylene glycol is less than 34%, while the redissolved microbubble solution has more uniform particle size distribution, the microbubbles show better film strength at the same ultrasonic frequency, and the endurance time is longer at high frequency.

The present application claims priority to the prior application with the patent application No. 202111296255.3 and entitled “MICROBUBBLE LYOPHILIZED PREPARATION FOR ULTRASONIC RADIOGRAPHY, CONTRAST AGENT, AND PREPARATION METHOD” filed to the China National Intellectual Property Administration on Nov. 3, 2021, which is incorporated herein by reference in its entirety.

### TECHNICAL FIELD

The present disclosure relates to the field of contrast agents, and in particular to a microbubble lyophilized preparation for ultrasonic radiography, a contrast agent, and a preparation method.

### BACKGROUND

Contrast agents are commonly used for enabling the imaging of human or animal organs and tissues, especially blood vessels and body cavities. The contrast agent is more or less dense than the surrounding tissues, and the resulting contrast is used for image display by the instrument.

Ultrasonic radiography not only enables the detection of small lesions that are difficult to discover with conventional ultrasonography but also has its unique advantages in interventional therapy and efficacy evaluation.

The current contrast agent for ultrasonic radiography comprises a suspension of nanobubbles or microbubbles dispersed in an aqueous medium, with a gas encapsulated in a soft membrane structure. The microbubbles have good resonance characteristics under the action of low sound pressure and can generate relatively strong harmonic signals, so that low-noise real-time harmonic images are obtained, which is beneficial to scanning sections of organs and tissues for a long time.

The suspension with gas microbubbles can be used as an effective ultrasound reflector due to the fact that dissolved gas escapes from the solution and becomes bubbles, thereby enhancing the echo signals of ultrasonic radiography. Generally, the suspension with gas microbubbles is obtained by reconstituting an ultrasonic contrast agent in the form of a lyophilized powder with a dispersant, for example, normal saline.

Chinese patent publication No. CN1088456A describes an injectable suspension of gas-filled microbubbles in an aqueous liquid carrier, which contains an amphiphilic compound, especially a phospholipid stabilizer, thereby preventing the microbubbles from collapsing due to time and pressure.

Chinese patent publication No. CN112165959A describes a lyophilized powder composition for the preparation of gas-filled microbubbles, which comprises a phospholipid and polyethylene glycol, wherein the polyethylene glycol needs to have a percentage of a folded polymer chain of greater than 34%. According to the patent document, if the percentage of the folded chain in the polyethylene glycol is too low, it will result in a deterioration of the quality of the lyophilized powder, whereby too many vials in the production process fail to pass the acceptability test in the production batch.

However, the storage stability of the lyophilized composition and the duration the microbubbles are present of the suspension of gas-filled microbubbles prepared therefrom, involved in the patent publications described above, respectively, are still in need of improvement.

### CONTENTS OF THE INVENTION

In order to improve the technical problems described above, the present disclosure provides a microbubble lyophilized preparation for ultrasonic radiography, which comprises polyethylene glycol and a surfactant, wherein the polyethylene glycol has a percentage of a folded polymer chain of less than 34%.

Preferably, the polyethylene glycol has a percentage of a folded polymer chain of less than 30%, more preferably, the polyethylene glycol has a percentage of a folded polymer chain of less than 25%, and further preferably, the polyethylene glycol has a percentage of a folded polymer chain of less than 20%, for example, 20%, 22%, 24%, 26%, 28%, 31%, 32%, 33%, or any point value in a range consisting of any two of the intermediate values. The percentage of the folded polymer chain of the polyethylene glycol can be determined by, for example, the DSC method described in CN112165959A, particularly the method described in Example 1 of the patent document.

According to an embodiment of the present disclosure, the polyethylene glycol has an average molecular weight of 4000-6000, and is preferably PEG4000 or PEG6000. The polyethylene glycol preferably has a melting point of 64-66° C., more preferably 65° C. The polyethylene glycol preferably has a density (25° C.) of 1.25-1.30 g/mL, more preferably 1.27 g/mL. The polyethylene glycol preferably has a refractive index of 1.465-1.470, more preferably 1.469. Preferably, the polyethylene glycol has a hydroxyl value of 26-32 mg KOH/g.

According to an embodiment of the present disclosure, the lyophilized preparation comprises a surfactant, preferably an amphiphilic surfactant, preferably a phospholipid and/or a phospholipid derivative. Preferably, the phospholipid comprises at least one of 1,2-diacyl-sn-glycero-3-phosphatidylcholine, diacylphosphatidylethanolamine, or phosphatidylethanolamine; preferably, the phospholipid derivative is at least one of 1,2-distearoyl-sn-glycero-3-phosphorylcholine, distearoylphosphatidylethanolamine, or 1,2-dipalmitoylphosphatidylglycerol sodium salt.

According to an embodiment of the present disclosure, the lyophilized preparation further comprises a fatty acid. The fatty acid can be a saturated or unsaturated fatty acid. Preferably, the fatty acid is selected from palmitic acid, oleic acid, and linoleic acid.

According to an embodiment of the present disclosure, the lyophilized preparation further comprises a surface charge modifier, wherein preferably, the surface charge modifier comprises at least one of an amino acid, trehalose, sodium alginate, chitosan and a derivative thereof, and sodium hydroxycellulose, for example, trehalose.

According to an embodiment of the present disclosure, the lyophilized preparation additionally comprises a pharmaceutically acceptable auxiliary material, for example, a thickener, an emulsifier, an antioxidant, a filler, a pH adjuster, a tonicity adjusting agent, a viscosity adjusting agent, and the like.

According to an embodiment of the present disclosure, the mass ratio of the polyethylene glycol to the surfactant is 40:1-85:1, preferably, the mass ratio of the polyethylene glycol to the surfactant is 45:1-70:1, and more preferably, the mass ratio of the polyethylene glycol to the surfactant is 50:1-60:1, for example, 42:1, 48:1, 52:1, 60:1, 65:1, 70:1, 80:1, or any value in a range consisting of any two of the point values.

According to an embodiment of the present disclosure, the mass ratio of the polyethylene glycol to the fatty acid is 275:1-1400:1, preferably, the mass ratio of the polyethylene glycol to the fatty acid is 300:1-1000:1, more preferably, the mass ratio of the polyethylene glycol to the fatty acid is 400:1-800:1, and further preferably, the mass ratio of the polyethylene glycol to the fatty acid is 400:1-600:1, for example, 300:1, 350:1, 400:1, 450:1, 500:1, 600:1, 700:1, 800:1, 900:1, 1000:1, 1100:1, 1200:1, 1300:1, or any value in a range of consisting of any two of the point values.

The present disclosure also provides a preparation method for the lyophilized preparation described above, which comprises the following steps:
dissolving the polyethylene glycol and the surfactant in a solvent, and lyophilizing the solution.

Preferably, lyophilizing the solution comprises freezing the solution and removing the solvent. According to the present disclosure, the solvent can dissolve the polyethylene glycol and the surfactant, and can be a mixed solvent. For example, the solvent is selected from a hydrocarbon, an alcohol, a ketone, and a combination thereof, preferably pentane, hexane, methanol, ethanol, propanol, isopropanol, n-butanol, sec-butanol, tert-butanol, acetone, methyl ethyl ketone, and the like.

According to an embodiment of the present disclosure, dissolving the polyethylene glycol and the surfactant in the solvent comprises: dissolving the surfactant in a solvent A and drying the solution to obtain a dried product, mixing the dried product with the polyethylene glycol, dissolving the mixture in a solvent B to obtain a mixed solution, and filling the mixed solution into a container.

Preferably, the solvent A and the solvent B are as defined above for the solvent. For example, the solvent A is a mixed solvent of hexane and ethanol, wherein preferably, the hexane and the ethanol are in a volume ratio of 8:2; for example, the solvent B is tert-butanol.

Preferably, the surfactant is as described above. For example, the surfactant is a mixture of 1,2-distearoyl-sn-glycero-3-phosphorylcholine (DSPC), dipalmitoylphosphatidylglycerol sodium salt (DPPG-Na), and palmitic acid (PA), wherein preferably, the mixture is dissolved in the solvent A to obtain a solution at a concentration of about 5 g/L.

Preferably, the mass ratio of the dried product to the polyethylene glycol is as defined above, and more preferably, the polyethylene glycol is PEG4000.

Preferably, the mixture is dissolved in the solvent B and mixed at a temperature of 45-65° C., more preferably at a temperature of 50-60° C., and further preferably at a temperature of 53-56° C. For example, the solvent B is preheated to 55° C. before the mixture is added to the solvent.

Preferably, the mixing is performed under stirring for a period of 10-30 min, more preferably for a period of 15-20 min, for example, about 15 min.

Preferably, before filling the mixed solution into the container, the method further comprises a step of leaving the mixed solution standing until the mixed solution reaches an equilibrium. More preferably, the mixed solution is left standing for a period of 0.5-4 h, further preferably for a period of 1-3 h, and further preferably for a period of 2-3 h, for example, about 1 h.

According to an embodiment of the present disclosure, the solution is lyophilized at a temperature of −30 to −50° C., preferably at a temperature of −40 to −50° C., for example, −45° C.

The present disclosure also provides a contrast agent, which comprises the lyophilized preparation according to the present disclosure and a gas.

According to an embodiment of the present disclosure, the lyophilized preparation and the gas are sealed in a container and are in contact with each other.

Preferably, the gas is at least one of an inert gas or sulfur hexafluoride, preferably sulfur hexafluoride; more preferably, the gas is biocompatible.

The present disclosure also provides a preparation method for the contrast agent described above, which comprises charging the gas into a container filled with the lyophilized preparation.

Preferably, the gas is charged until the container is saturated with the gas.

Preferably, the gas is charged under atmospheric pressure, preferably for a period of 0.5 h to 2 h, and more preferably for a period of 1 h to 1.5 h, for example, 1 h.

The present disclosure also provides a vial containing a contrast agent, which comprises the lyophilized preparation according to the present disclosure and a gas.

The present disclosure also provides a use method for the contrast agent described above, which comprises dispersing the contrast agent described above in a physiologically acceptable solution to obtain a suspension of gas-filled microbubbles.

Preferably, the physiologically acceptable solution is a normal saline solution.

### Beneficial Effects

The inventors have surprisingly found that when the percentage of the folded polymer chain of the polyethylene glycol is less than 34%, the microbubble solution obtained after re-dissolution has a more uniform particle size distribution, and the microbubbles exhibit better membrane strength at the same ultrasonic frequency and have a longer endurance time at a high frequency.

### BRIEF DESCRIPTION OF THE DRAWINGS

FIG. 1 is a graph showing the particle size distribution of the microbubbles of Comparative Example 1 after re-dissolution;

FIG. 2 is a graph showing the particle size distribution of the microbubbles of Example 1 after re-dissolution;

FIG. 3 is a picture showing a sample of Comparative Example 1 after re-dissolution;

FIG. 4 is a picture showing a sample of Example 1 after re-dissolution.

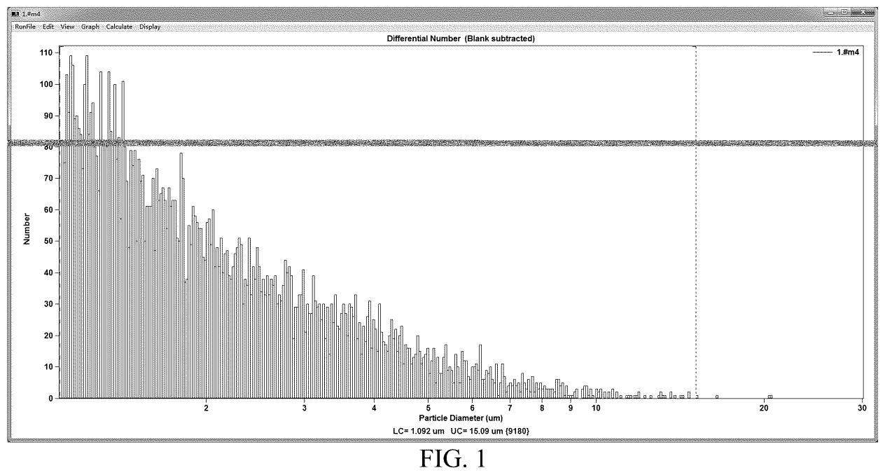

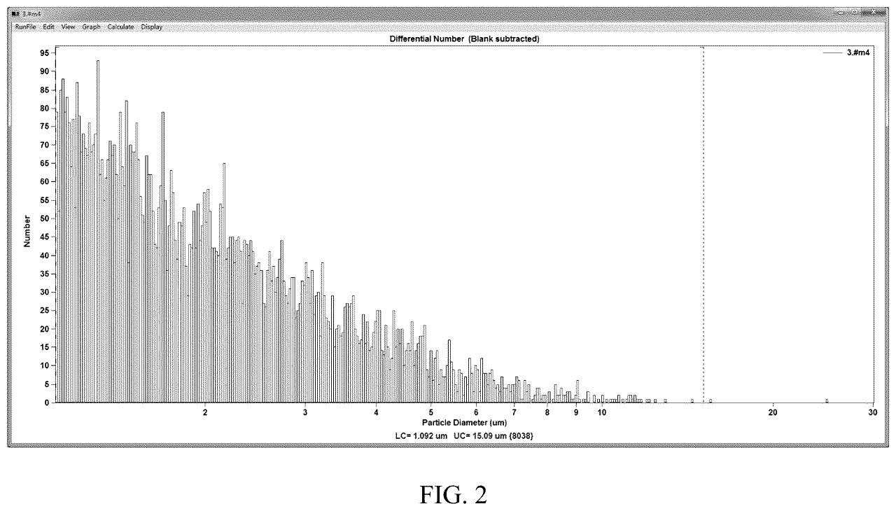

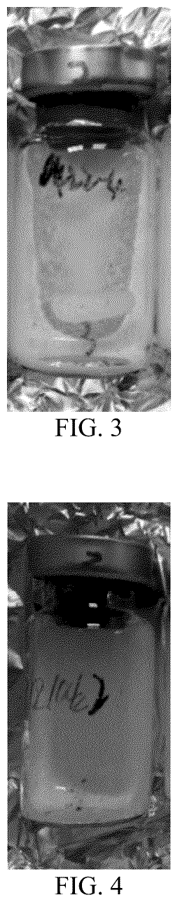

### MODE FOR CARRYING OUT THE INVENTION

The compounds of the present disclosure, the preparation method therefor, and use thereof are described in detail with reference to the following specific examples. It should be understood that the following examples are merely exemplary illustrations and explanations of the present disclosure, and should not be construed as limiting the protection scope of the present disclosure. All techniques implemented based on the content of the present disclosure described above are encompassed within the protection scope of the present disclosure.

Unless otherwise stated, the starting materials and reagents used in the following examples are all commercially available products, or can be prepared by using known methods.

### Example 1

DSPC (1,2-distearoyl-sn-glycero-3-phosphorylcholine), DPPG-Na (dipalmitoylphosphatidylglycerol sodium salt), and palmitic acid (PA) were prepared as a dried product A according to a weight ratio of 6.2:6.2:1. The dried product A and PEG4000 having a percentage of a folded chain of 26% were dissolved, according to a weight ratio of 0.015:1, in tert-butanol preheated for 5 min at a temperature of 55° C., and stirred for dissolution at 50° C. for 15 min. After the system was equilibrated for 60 min, the mixed solution was filled into a vial (containing about 25 mg of the mixed solution according to the corresponding volume), and the vial was rapidly cooled at −45° C. and lyophilized to remove the solvent. At the end of the lyophilization, SF₆ was charged into the vial for 30 min under atmospheric pressure until the vial was saturated with SF₆.

### Comparative Example 1

The comparative example was performed under the same conditions as Example 1 except that PEG4000 having a percentage of a folded chain of more than 34% was used. The PEG4000 used in this example was manufactured by Bracco International B.V, with the batch No. 20A093A F0608Z.

### Test Example 1. Determination of Particle Size and Particle Size Distribution of Microbubbles

A 256-channel Coulter microparticle counter with a data processing system was used. The instrument was repeatedly washed with a microparticle-free blank solution (0.9% sodium chloride solution), and then the microparticles of the blank solution was determined. The number of particles with a particle size of no more than 0.7 μm should not exceed 300.

95 mg of each of the samples in Example 1 and Comparative Example 1 was taken and dissolved by adding 5 mL of 0.9% sodium chloride solution. The mixture was strongly shaken for 20 s to obtain a sample solution. 0.1 mL of the sample solution was precisely pipetted into 100 mL of microparticle-free blank solution (0.9% sodium chloride solution) (placed in a measuring cup), and the mixture was left standing for 5-6 s, stirred, and determined for the particle size of microbubbles.

After the determination, the measuring cup was taken out and sonicated for 10 s to destroy the bubbles, and the mixture was determined again, with the result being used as a blank background count. The determination result for the blank background solution was subtracted from each of the determination results for the solutions of Example 1 and Comparative Example 1, and determination data was read from the curve. A duplicate determination was performed on three sets of samples, and the mean value was taken as the determination result.

As can be seen from Table 1, in which the determination results were shown, the proportion of bubbles having a particle size of more than 15 μm was 0.04% and the proportion of bubbles having a particle size of less than 8 μm was 98.79% after the sample of Comparative Example 1 was re-dissolution; the bubbles all had a particle size of less than 8 μm after the sample of Example 1 was re-dissolution, which indicated that the sample of the example of the present disclosure had a more uniform particle size distribution after being re-dissolved.

TABLE 1
Determination results:

| | Comparative | |
|---|---|---|
| Sample | Example 1 | Example 1 |
| Mean concentration (bubbles/mL) | 1.84 × 10⁸ | 1.59 × 10⁸ |
| Proportion of <8 μm bubbles | 98.79% | 99.12% |
| Proportion of >15 μm bubbles | 0.04% | 0 |

As can be seen from Table 2, in which the test data for bubbles in the solution obtained after re-dissolution of contrast agents containing PEG4000 with different folding rates prepared by the method of Example 1 was shown, the proportion of bubbles with a particle size of less than 8 μm in the solution obtained after re-dissolution of the contrast agent prepared by using PEG4000 with a folding rate of 38% was 98.79%, and the proportion of the bubbles with a particle size of less than 8 μm gradually increased with the decrease of the folding rate of the PEG4000 used. Under the condition that the ultrasonic frequency was 7 MHz, the endurance time of the bubbles gradually increased with the decrease of the folding rate; under the condition that the ultrasonic frequency was 3.5 MHz, the endurance time of the bubbles also gradually increased with the decrease of the folding rate.

TABLE 2
Membrane strength of microbubbles after re-dissolution
of contrast agents prepared from PEG4000 with different
folding rates under ultrasonic wave

| | | | Endurance time | Endurance time |
|---|---|---|---|---|
| | Mean | Proportion | at high ultra- | at low ultra- |
| Folding | concentra- | of <8 μm | sonic frequency | sonic frequency |
| rate | tion | bubbles | (7 MHz) | (3.5 MHz) |
| 12% | 1.39 × 10⁸ | 99.63% | 380 s | 450 s |
| 18% | 1.54 × 10⁸ | 99.25% | 340 s | 420 s |
| 26% | 1.59 × 10⁸ | 99.12% | 280 s | 380 s |
| 32% | 1.61 × 10⁸ | 99.07% | 190 s | 270 s |
| 38% | 1.84 × 10⁸ | 98.79% | 110 s | 250 s |

The embodiments of the present disclosure have been described above. However, the present disclosure is not limited to the embodiments described above. Any modification, equivalent, improvement, and the like made without departing from the spirit and principle of the present disclosure shall fall within the protection scope of the present disclosure.

1. A microbubble lyophilized preparation for ultrasonic radiography, comprising polyethylene glycol and a surfactant, wherein the polyethylene glycol has a percentage of a folded polymer chain of less than 34%.

2. The microbubble lyophilized preparation for ultrasonic radiography according to claim 1, wherein the polyethylene glycol has a percentage of a folded polymer chain of less than 30%, preferably, the polyethylene glycol has a percentage of a folded polymer chain of less than 25%, and more preferably, the polyethylene glycol has a percentage of a folded polymer chain of less than 20%;
preferably, the polyethylene glycol has an average molecular weight of 4000-6000, and is preferably PEG4000 or PEG6000;
preferably, the polyethylene glycol has a melting point of 64-66° C., a density (25° C.) of 1.25-1.30 g/mL, and a refractive index of 1.465-1.470, and more preferably, the polyethylene glycol has a hydroxyl value of 26-32 mg KOH/g.

3. The microbubble lyophilized preparation for ultrasonic radiography according to claim 1, comprising a surfactant, preferably an amphiphilic surfactant, wherein
preferably, the mass ratio of the polyethylene glycol to the surfactant is 40:1-85:1, more preferably, the mass ratio of the polyethylene glycol to the surfactant is 45:1-70:1, and further preferably, the mass ratio of the polyethylene glycol to the surfactant is 50:1-60:1.

4. The microbubble lyophilized preparation for ultrasonic radiography according to claim 1, further comprising a fatty acid, wherein the fatty acid can be a saturated or unsaturated fatty acid;
preferably, the mass ratio of the polyethylene glycol to the fatty acid is 275:1-1400:1, preferably, the mass ratio of the polyethylene glycol to the fatty acid is 300:1-1000:1, more preferably, the mass ratio of the polyethylene glycol to the fatty acid is 400:1-800:1, and further preferably, the mass ratio of the polyethylene glycol to the fatty acid is 400:1-600:1.

5. The microbubble lyophilized preparation for ultrasonic radiography according to claim 1, further comprising a surface charge modifier, wherein
preferably, the lyophilized preparation additionally comprises a pharmaceutically acceptable auxiliary material.

6. A preparation method for the lyophilized preparation according to claim 1, comprising the following steps:
dissolving the polyethylene glycol and the surfactant in a solvent, and lyophilizing the solution, wherein
preferably, lyophilizing the solution comprises freezing the solution and removing the solvent;
preferably, the solvent can dissolve the polyethylene glycol and the surfactant;
according to an embodiment of the present disclosure, dissolving the polyethylene glycol and the surfactant in the solvent comprises: dissolving the surfactant in a solvent A and drying the solution to obtain a dried product, mixing the dried product with the polyethylene glycol, dissolving the mixture in a solvent B to obtain a mixed solution, and filling the mixed solution into a container.

7. The preparation method for the lyophilized preparation according to claim 6, comprising a step of dissolving the mixture in the solvent B and performing mixing at a temperature of 45-65° C., more preferably at a temperature of 50-60° C., and further preferably at a temperature of 53-56° C., wherein
preferably, the mixing is performed under stirring for a period of 10-30 min, more preferably for a period of 15-20 min;
preferably, before filling the mixed solution into the container, the method further comprises a step of leaving the mixed solution standing until the mixed solution reaches an equilibrium; more preferably, the mixed solution is left standing for a period of 0.5-4 h, further preferably for a period of 1-3 h, and still further preferably for a period of 2-3 h;
preferably, the solution is lyophilized at a temperature of −30 to −50° C., preferably at a temperature of −40 to −50° C.

8. A contrast agent comprising a gas and the lyophilized preparation according to claim 1, wherein
preferably, the lyophilized preparation and the gas are sealed in a container and are in contact with each other;
preferably, the gas is at least one of an inert gas or sulfur hexafluoride, preferably sulfur hexafluoride; more preferably, the gas is biocompatible.

9. A preparation method for the contrast agent according to claim 8, comprising charging a gas into a container filled with the lyophilized preparation, wherein
preferably, the gas is charged until the container is saturated with the gas;
preferably, the gas is charged under atmospheric pressure, more preferably for a period of 0.5-2 h, and further preferably for a period of 1-1.5 h.

10. A use method for the contrast agent according to claim 8, comprising dispersing the contrast agent in a physiologically acceptable solution to obtain a suspension of gas-filled microbubbles, wherein
preferably, the physiologically acceptable solution is a normal saline solution.
